SEQUENCE LISTING (16 sequences)

SEQ ID NO: 1 - DNA, 22 bp, Artificial Sequence
  Forward primer sequence
gcatttgtcctttgtttgacat

SEQ ID NO: 2 - DNA, 22 bp, Artificial Sequence
  Reverse primer sequence
tttttcgcgaagtgttctctac

SEQ ID NO: 3 - DNA, 24 bp, Artificial Sequence
  Forward primer sequence
gtcaaatttttgatctttctctct

SEQ ID NO: 4 - DNA, 24 bp, Artificial Sequence
  Reverse primer sequence
ggagagacagagtacagtagaggt

SEQ ID NO: 5 - DNA, 24 bp, Artificial Sequence
  Forward primer sequence
aatttaggttaaggttttgatttg

SEQ ID NO: 6 - DNA, 24 bp, Artificial Sequence
  Reverse primer sequence
agagagaataggttacagcatcat

SEQ ID NO: 7 - DNA, 22 bp, Artificial Sequence
  Forward primer sequence
tgtcactgcttacagaaacaat

SEQ ID NO: 8 - DNA, 24 bp, Artificial Sequence
  Reverse primer sequence
tatgacgagattgtataagttgct

SEQ ID NO: 9 - DNA, 23 bp, Artificial Sequence
  Forward primer sequence
ccaagtcagtttggtagtaattc

SEQ ID NO: 10 - DNA, 23 bp, Artificial Sequence
  Reverse primer sequence
agtgcaacttttacttgctatgt

SEQ ID NO: 11 - DNA, 24 bp, Artificial Sequence
  Forward primer sequence
cttaagtcacaaagacaatgaaat

SEQ ID NO: 12 - DNA, 24 bp, Artificial Sequence
  Reverse primer sequence
aagagagttcagatttatctttgc

SEQ ID NO: 13 - DNA, 20 bp, Artificial Sequence
  Forward primer sequence
agcactcatagtccttgcac

SEQ ID NO: 14 - DNA, 24 bp, Artificial Sequence
  Reverse primer sequence
gttaaaacgaagaagatacaacaa

SEQ ID NO: 15 - DNA, 24 bp, Artificial Sequence
  Forward primer sequence
gaaagagataaaagatggttcaat

SEQ ID NO: 16 - DNA, 22 bp, Artificial Sequence
  Reverse primer sequence
cttatagagtaagcccaccatc

(19) United States
(12) Plant Patent Application Publication
(10) Pub. No.: US 2020/0323118 P1
(43) Pub. Date: Oct. 8, 2020

(54) Cornus Kousa Tree Named 'Melissa's Mountain Snowfall'

(50) ;

(71) Applicant: UNIVERSITY OF TENNESSEE RESEARCH FOUNDATION, KNOXVILLE, TN (US)

(72) Inventors: Robert N. Trigiano, Knoxville, TN (US); Sarah Lynn Boggess, Knoxville, TN (US)

(21) Appl. No.: 16/602,154

(22) Filed: Aug. 15, 2019

Related U.S. Application Data
(60) Provisional application No. 62/830,688, filed on Apr. 8, 2019.

(51) Int. Cl. A01H 6/00; A01H 5/02

(52) U.S. Cl. CPC A01H 5/02; A01H 6/00
USPC PLT/220

(57) ABSTRACT

A new and distinct cultivar of flowering dogwood tree, which has fused bracts is provided. This dogwood tree is botanically known as Cornus kousa and referred to by the following cultivar name: ‘Melissa's Mountain Snowfall’.

### CROSS-REFERENCE TO A RELATED APPLICATION

This application claims the benefit of U.S. Provisional Application Ser. No. 62/830,688, filed Apr. 8, 2019, the disclosure of which is hereby incorporated by reference in its entirety, including all figures, tables and drawings.

This invention was made with Government support under Contract No. NACA-58-6062-6 awarded by the U.S. Department of Agriculture. The Government has certain rights in the invention.

### BACKGROUND OF THE INVENTION

The present invention relates to a new and distinct dogwood cultivar, which has fused bracts. This dogwood is botanically known as Cornus kousa and hereinafter referred to by the following cultivar name: ‘Melissa's Mountain Snowfall’.

This new dogwood cultivar was discovered in a planting of seedlings in the University of Tennessee Arboretum in Oak Ridge, Tenn. ‘Melissa's Mountain Snowfall’ is a half-sibling of ‘Pam's Mountain Bouquet’ (U.S. Plant Pat. No. 25,575; Wadl et al., 2014, HortScience 49(9):1230-1233). Asexual reproduction of ‘Melissa's Mountain Snowfall’ has shown that the unique features of this new dogwood cultivar are stable and reproduced true-to-type in successive vegetative generations.

### BRIEF DESCRIPTION OF THE DRAWINGS

FIG. 1. Photograph of ‘Melissa's Mountain Snowfall’. Colors in the photograph may differ from actual colors due to lighting and light reflectance.

FIG. 2. Photograph of enlarged view of bracts on ‘Melissa's Mountain Snowfall’.

FIG. 3. Photograph of the unripe fruit of “Melissa's Mountain Snowfall.” Also shown are the paper collars of the dried bracts that remain on the petioles and around the fruit.

FIG. 4. Photograph of the ripe fruit of ‘Melissa's Mountain Snowfall’. FIG. 5. Photograph showing the exfoliating bark on the trunk of older specimens of ‘Melissa's Mountain Snowfall’.

### DETAILED DESCRIPTION OF THE NEW VARIETY

A new and distinct cultivar of flowering dogwood having fused bracts is provided. This dogwood tree cultivar is botanically known as Cornus kousa and referred to by the cultivar name: ‘Melissa's Mountain Snowfall’. This cultivar exhibits insect resistance and disease resistance, particularly to powdery mildew caused by Erisphe pulchra. Dogwood anthracnose caused by Discula destructiva has never been observed on ‘Melissa's Mountain Snowfall’.

This new and distinct dogwood tree cultivar was discovered in a planting of seedlings within the Arboretum at the University of Tennessee located in Oak Ridge, Tenn. The subject dogwood tree cultivar is a half-sibling of the Cornus kousa dogwood cultivar known as ‘Pam's Mountain Bouquet’. Table 1 shows the observed phenotypic similarities and differences between the two cultivars.

TABLE 1
General phenotypic differences between the dogwood cultivars ‘Melissa's
Mountain Snowfall’ and ‘Pam's Mountain Bouquet’. (** = Key differences)

| ‘Melissa's Mountain Snowfall’ | ‘Pam's Mountain Bouquet’ |
|---|---|
| About 80% of all bracts on | About 82% of all bracts on |
| the cultivar exhibit some | the cultivar exhibit some |
| degree of fusion | degree of fusion |
| Resistance to Disease and | Resistance to Disease and |
| Insect Damage | Insect Damage |
| Exfoliating Bark in older specimens** | No exfoliating bark |
| Inverted pyramidal growth habit** | Spreading growth habit |
| Multiple leaders** | Single leader |
| Six meters in height** | 3-4 meters in height |

In addition to the phenotypic differences listed above, it has also been observed that the alleles of the two cultivars differ at 5 of 8 selected loci. Asexual reproduction of ‘Melissa's Mountain Snowfall’ by grafting of axillary buds onto seedling rootstocks has shown that the unique features of this new dogwood cultivar are stable and reproduced true-to-type in successive generations.

### DETAILED BOTANICAL DESCRIPTION

The following observations, measurements and comparisons describe the cultivar ‘Melissa's Mountain Snowfall’ grown in Oak Ridge, Tenn. Trees used for this description were about thirty (30) years old. Plant hardiness is expected to be zones 3-9. The color characteristic descriptions use color references to The Royal Horticultural Society (R.H.S.) Colour Chart, The Royal Horticultural Society, London, UK, 4^{th} Edition, 2001, except where general terms of ordinary dictionary significance are used. It has been determined that alleles differ at 5 of 8 loci shared by ‘Melissa's Mountain Snowfall’ and ‘Pam's Mountain Bouquet’, as shown in Table 2.

TABLE 2
Allelic Comparison of ‘Melissa's Mountain Snowfall’ and ‘Pam's
Mountain Bouquet’ at specified loci

| | ‘Melissa's Mountain | ‘Pam's Mountain |
|---|---|---|
| | Snowfall’ (bp size | Bouquet’ (bp size |
| Locus | for each allele) | for each allele) |
| CK005* | 228:228 | 222:247 |
| CK072* | 113:122 | 113:117 |
| CK058* | 152:152 | 148:148 |
| CK031 | 140:140 | 140:140 |
| CK040* | 102:102 | 94:94 |
| CK029 | 90:102 | 90:102 |
| CK015* | 119:122 | 130:136 |
| CK047 | 128:128 | 128:128 |

Table 3 indicates the primer sequences and microsatellite markers (or single sequence repeats—SSR) in ‘Melissa's Mountain Snowfall’ compared with the same microsatellite markers (SSR) in ‘Pam's Mountain Bouquet.’ Those loci indicated with an asterisk (*) differ between the two cultivars:

TABLE 3
Primer Sequences and Microsatellite markers
compared between ‘Melissa's Mountain Snowfall’
and ‘Pam's Mountain Bouquet’

| GenBank | | Microsatellite Repeat Sequences | |
|---|---|---|---|
| Acces- | | | Repeat |
| sion No. | Locus | Primer Sequence (5′-3′) | Motif |
| EU544308 | CK005* | F: GCATTTGTCCTTTGTTTGACAT | (AC)₂₀ |
| | | (SEQ ID 1) | |
| | | R: TTTTTCGCGAAGTGTTCTCTAC | |
| | | (SEQ ID 2) | |
| EU125523 | CK015* | F: GTCAAATTTTTGATCTTTCTCTCT | (CT)₁₀ |
| | | (SEQ ID 3) | |
| | | R: GGAGAGACAGAGTACAGTAGAGGT | |
| | | (SEQ ID 4) | |
| EU125524 | CK029 | F: AATTTAGGTTAAGGTTTTGATTTG | (TC)₈ |
| | | (SEQ ID 5) | |
| | | R: AGAGAGAATAGGTTACAGCATCAT | |
| | | (SEQ ID 6) | |
| EU125525 | CK031 | F: TGTCACTGCTTACAGAAACAAT | (CT)₇ |
| | | (SEQ ID 7) | |
| | | R: TATGACGAGATTGTATAAGTTGCT | |
| | | (SEQ ID 8) | |
| EU125526 | CK040* | F: CCAAGTCAGTTTGGTAGTAATTC | (GT)₁₆ |
| | | (SEQ ID 9) | |
| | | R: AGTGCAACTTTTACTTGCTATGT | |
| | | (SEQ ID 10) | |
| EU544309 | CK058* | F: CTTAAGTCACAAAGACAATGAAAT | (GT)₁₀ |
| | | (SEQ ID 11) | |
| | | R: AAGAGAGTTCAGATTTATCTTTGC | |
| | | (SEQ ID 12) | |
| EU544312 | CK072* | F: AGCACTCATAGTCCTTGCAC | (GT)₁₀ |
| | | (SEQ ID 13) | |
| | | R: GTTAAAACGAAGAAGATACAACAA | |
| | | (SEQ ID 14) | |
| EU125528 | CK047 | F: GAAAGAGATAAAAGATGGTTCAAT | (AC)₆ |
| | | (SEQ ID 15) | |
| | | R: CTTATAGAGTAAGCCCACCATC | |
| | | (SEQ ID 16) | |

The cultivar ‘Melissa's Mountain Snowfall’ has some similarity in phenotypic characteristics to the cultivar ‘Pam's Mountain Bouquet’ (Wadl et al., 2014). The following Table 4 provides a comparison of each cultivar for those characteristics that have been observed. Measurements are provided as an average (with ranges also provided as indicated):

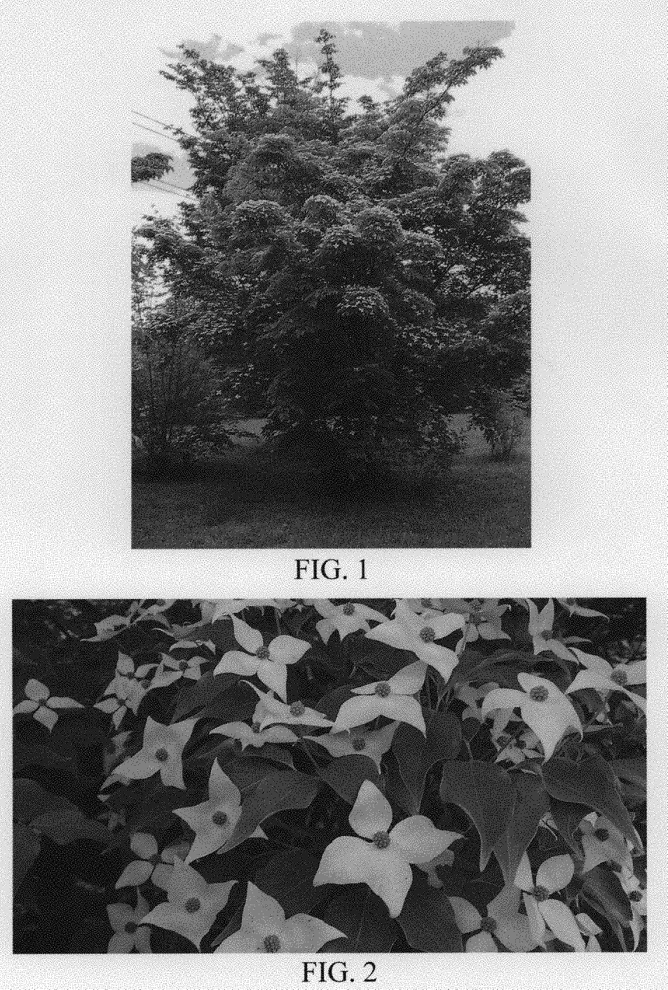

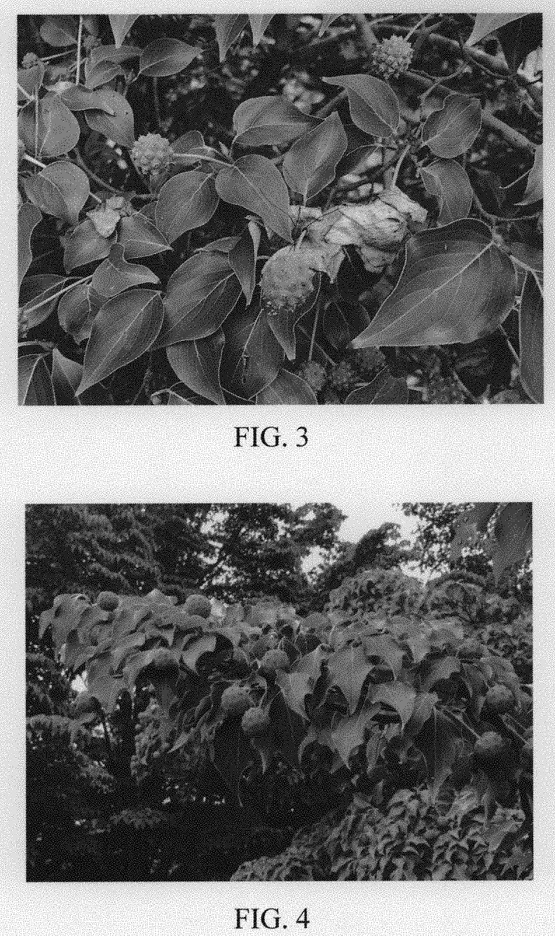

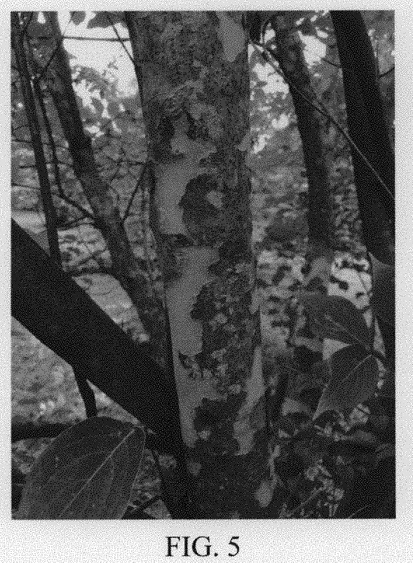

TABLE 4
Characteristics of ‘Melissa's Mountain Snowfall’and
Pam's Mountain Bouquet′
Color Descriptions are based upon the
Royal Horticultural Society's (RHS) colour chart, 4th Edition 2001.

| | | ‘Melissa's Mountain | ‘Pam's Mountain |
|---|---|---|---|
| | Character | Snowfall’ | Bouquet’ |
| 1 | Tree form | Inverted pyramidal | spreading |
| | (observation) | | |
| 2 | Tree height | 5-6 meters | low |
| | (observation) | | (about 3-4 |
| | | | meters; spread |
| | | | about 4 -5 |
| | | | meters, and |
| | | | dependent on age |
| | | | and environment) |
| 3 | Branch thickness | Not observed | medium |
| | (measurement) | | (age dependent) |
| | Thickness in the | | |
| | middle portion of | | |
| | a plant | | |
| 4 | Color of current | Not observed | Green |
| | Shoot | | 143B |
| | (observation) | | |
| | current shoot | | |
| | color in the | | |
| | middle portion of | | |
| | a plant | | |
| 5 | Branch color | Mixture of 156A, | Greyed; Green |
| | (observation) | 197B, and 198B | 198B |
| | current branch | | |
| | color in the | | |
| | middle portion of | | |
| | a plant second | | |
| | year+ | | |
| 6 | Dark spots on | Not observed | Absent |
| | Branch | | |
| | (observation) | | |
| | presence of dark | | |
| | spots on the | | |
| | branch | | |
| 7 | Branching | High | High |
| | (observation) | | |
| | density of | | |
| | branching | | |
| 8 | Internode length | Not observed | Short |
| | (measurement) | | |
| | Internode length | | |
| | in the middle | | |
| | portion of a plant | | |
| 9 | Whole shape of | Obovate | Obovate |
| | leaves (observation) | | |
| | see FIG. 1 whole | | |
| | shape of a leaf in | | |
| | the middle portion | | |
| | of a plant | | |
| 10 | Shape of leaf | Acuminate | Acuminate |
| | tip (observation) | | |
| | see FIG. 2 Tip | | |
| | shape of a leaf in | | |
| | the middle portion | | |
| | of a plant | | |
| 11 | Shape of leaf | Truncate | Truncate |
| | Base (observation) | | |
| | see FIG. 2 | | |
| | Base shape of a | | |
| | leaf in the middle | | |
| | portion of a plant | | |
| 12 | Shape of leaf | Entire | Entire |
| | Margin (observation) | | |
| | shape of a leaf | | |
| | margin in the | | |
| | middle portion of | | |
| | a plant | | |
| 13 | Leaf rolling | Not observed | Rolling inward |
| | (observation) see | | |
| | FIG. 4 | | |
| 14 | Leaf curvature | Not observed | Flat |
| | (observation) see | | |
| | FIG. 5 | | |
| 15 | Leaf margin | Not observed | None |
| | Undulation (observation) | | |
| 16 | Leaf length | Averages 87.1 mm | Long |
| | (measurement) | | (about 100-140 |
| | Length from the | | m) |
| | tip to the base of | | |
| | mature leaf | | |
| 17 | Leaf width | Averages 44.4 mm | Narrow |
| | (measurement) | | (about 40-50 |
| | The maximum | | mm) |
| | width of mature | | |
| | leaf | | |
| 18 | Leaf thickness | Medium | Medium |
| | (observation) | | |
| | Thickness of | | |
| | mature leaf | | |
| 19 | Bud color (observation) | 138B, unopened; | Grayish green |
| | Color of bud just | 132D, opened | 179A |
| | after sprouting | | |
| 20 | Immature leaf | Not observed | Light Green |
| | color (observation) | | 135B |
| 21 | Presence of | Absent | Absent |
| | anthocyanin | | |
| | (observation) | | |
| | Coloration by | | |
| | anthocyanin on | | |
| | the immature leaf | | |
| | upperside | | |
| 22 | Color of leaf | 143A | Green |
| | upperside (observation) | | 143B |
| | Color of mature | | |
| | leaf upperside | | |
| 23 | Color of leaf | 143C | Light Green |
| | lowerside (observation) | | 146B |
| | Color of mature | | |
| | leaf lowerside | | |
| 24 | Seasonal change | Changed | Changed |
| | of a mature leaf | | |
| | (observation) | | |
| 25 | Color of leaves in | Red | Red |
| | autumn (observation) | 10C −46A | 10C −46A |
| 26 | Leaf variegation | Not variegated | Not variegated |
| | (observation)Variegation | | |
| | on | | |
| | leaf upperside | | |
| 27 | Variegation | Not observed | NA |
| | pattern (observation); | | |
| | Pattern of | | |
| | variegation on a | | |
| | leaf upperside | | |
| 28 | Variegation color | Not observed | NA |
| | (observation) | | |
| 29 | seasonal change | Not observed | NA |
| | of variegation | | |
| | color (observation) | | |
| 30 | Hair on leaf | None | None |
| | upperside (observation) | | |
| | hair density on a | | |
| | mature leaf | | |
| | upperside | | |
| 31 | Hair on leaf | None | None |
| | lowerside (observation) | | |
| | hair density on a | | |
| | mature leaf | | |
| | lowerside | | |
| 32 | Petiole length | Average about 10.4 | Short |
| | (measurement) Length | mm; unequal | (about 15-25 |
| | from | at base, about 5-7 | mm) |
| | the base of blade | mm longer | |
| | to the base petiole | on one side | |
| 33 | Petiole width | Medium (<7 mm) | Medium |
| | (measurement) | | (<8 mm) |
| | The maximum | | |
| | width of a mature | | |
| | leaf petiole | | |
| 34 | Petiole color | Green 143A | Green |
| | (observation) | | 143B |
| 35 | Inflorescence type | Umbel | Umbel |
| | (observation) | | |
| 36 | Inflorescence | Upright | Upright |
| | direction (observation) | | |
| 37 | Inflorescence | Average about 31.7 | Medium |
| | diameter (observation) | | (diagonal mean |
| | | | length = 74 mm.; |
| | | | mean width = |
| | | | 53 mm) |
| 38 | Flower diameter | Not observed | Small |
| | (measurement) | | |
| 39 | Floret diameter | Not observed | Small |
| | (measurement) | | |
| 40 | Floret color | Yellow | Yellow |
| | (observation) | 150A | 150C |
| 41 | Bract type (observation) | 80% are fused, but | 83% are fused, |
| | | variable (See Table) | but variable |
| | | | (See Table 3) |
| 42 | Uniformity of | Not uniform | Not uniform |
| | bract size (observation) | | |
| 43 | Bract overlapping | No overlap of | No overlap of |
| | (observation) | unfused bracts | unfused |
| | | | bracts |
| 44 | Bract orientation | Recurved, Reflexed, | Recurved, |
| | (observation) | or Flat | Reflexed, or Flat |
| 45 | Bract rolling | Varies (may roll | Varies (may roll |
| | (observation) | inward or outward | inward or |
| | | | outward |
| 46 | Degree of bract | Medium | strong |
| | rolling (observation) | | |
| 47 | Bract curvature | Varies | Varies |
| | (observation) | (can be recurved, | (can be recurved, |
| | | flat, or reflexed) | flat, or reflexed) |
| 48 | Bract twisting | None | None |
| | (observation) | | |
| 49 | Whole shape of | Ovate | Ovate |
| | bracts (observation) | | |
| 50 | Shape of bract | Acuminate | Acuminate |
| | apex (observation) | | |
| 51 | Unfused bract length | Inner Bract Average | Medium |
| | (measurement) | 48 mm; Outer Bract | |
| | | Average 43 mm | |
| 52 | Unfused Bract width | Inner bract Average | |
| | (measurement) | 27 mm; outer bract | |
| | | average 28 mm. | |
| 53 | Number of bracts | 4 FUSED; Diameter | FUSED, but 4 |
| | (measurement) | average 89.5 mm | |
| 54 | Bract color | 157B | 155A |
| | (measurement) | | (immature: |
| | | | 157A) |
| 55 | Bract variegation | Not variegated | Not variegated |
| | (observation) | | |
| 56 | Variegation | NA | NA |
| | pattern (observation) | | |
| 57 | Variegation color | NA | NA |
| | (measurement) | | |
| 58 | Pistil color (observation) | Yellow green Not | Yellow green |
| | | coded | Not coded |
| 59 | Stigma color | Dark Green | Dark Green |
| | (observation) | (Not Coded) | (Not Coded) |
| 60 | Peduncle | Medium | Medium |
| | thickness | | |
| | (measurement) | | |
| 61 | Peduncle length | Average 69 mm | Long |
| | (measurement) | | (mean of 68 mm) |
| 62 | Peduncle color | Yellow green 143C | Yellow green |
| | (observation) | | 144B |
| 63 | Fruit shape (observation) | Globose | Globose |
| 64 | Fruit length | About 28.7-29.3 mm | Medium |
| | (measurement) | | (about 40 mm) |
| 65 | Fruit width | About 28.7-29.3 | Medium |
| | (measurement) | mm | (about 4.0 mm) |
| 66 | Fruit color (observation) | 134N, Fall; 60D- | Unripe:143B; |
| | | 61A, when ripe | Ripe 33B to 43A. |
| | | | Highly variable |
| | | | depending on |
| | | | ripeness |
| 67 | Fragrance (observation) | Absent | Absent |
| 68 | Seed fertility | Not observed | High |
| | (observation) | | |
| 69 | Time to the first | Medium | Medium |
| | flowering (observation) | (April-mid-May) | (April-mid-May) |
| 70 | Blooming habit | Prolific | Many |
| | (observation) | | |
| 71 | Flowering season | One season | One season |
| | (observation) | | flowering |
| 72 | Flowering time | About 5-6 weeks | About 5-6 weeks |
| | (observation) | | |
| 73 | Deciduous or | Deciduous | Deciduous |
| | evergreen (observation) | | |
| 74 | Cold hardiness | To −20C | Medium |
| | (observation) | | (to −20° C.-no |
| | | | effect) |
| 75 | Heat tolerance | Strong | Strong |
| | (observation) | (to 40° C.-no effect) | (to 40° C.-no |
| | | | effect) |
| 76 | Pest resistance | No specific pests | Strong |
| | (observation) | noted some spots of | (no specific pests |
| | | brown anthracnose, | noted; |
| | | 160A | |
| 77 | Disease resistance | Strong resistant to | Strong resistant |
| | (observation) | dogwood | to dogwood |
| | | anthracnose and | anthracnose and |
| | | powdery mildew: | powdery mildew |
| | | some spot | some spot |
| | | anthracnose | anthracnose on |
| | | especially on bracts | especially on |
| | | | bracts |
| 78 | Bark color | exfoliating bark | Grayed-Green |
| | | 177b and 143C; | 198B |
| | | exfoliated areas | |
| | | 199C-D | |
| 79 | Bark texture | Exfoliating | Smooth |
| 80 | Angle of emerging | 20° C.-35° C.from | 20° C.-30° C. from |
| | branches | vertical stem | vertical stem |
| 81 | Time to first leaf bud | Mid-to late-April | Mid- to late-April |
| | burst | | |
| 82 | Leaf Vein color (bottom | 145B | Green-Greyed |
| | side) | | 192A |
| 83 | Immature Leaf color | Similar to fully | Similar to fully |
| | | expanded leaf color | expanded leaf |
| | | | color |
| 84 | Bract base | Truncate | Truncate |
| 85 | Bract margin | Entire | Entire |
| 86 | Vestiture | Puberulous, | Puberulous, |
| | | reticulate | reticulate |
| 87 | Flower/ | Mean =31 | Mean = 34 |
| | inflorescensce number | | |
| 88 | Seed shape | Flattened along | Flattened along |
| | | length | length |
| 89 | Seed color | Greyed Yellow | Greyed Yellow |
| | | 162D | 162D |
| 90 | Seed number | 0-17 per fruit | 0-17 per fruit |
| 91 | Bloom duration | 3-5 weeks | 3-5 weeks |
| | | (dried, dead bracts | (dried, dead |
| | | are retained as a | bracts are |
| | | “collar”on peduncle | retained as a |
| | | until fruit fall in | “collar”on |
| | | Autumn) | peduncle until |
| | | | fruit fall in |
| | | | Autumn) |
| 92 | Time of fruit ripening | Begins in mid- | Begins mid-to |
| | | August and Ripe in | late-August |
| | | October | through October |
| 93 | Trunk diameter (at base) | Multiple stem | 18 cm at 15 years |
| | | variable. About 10- | of age |
| | | 14 cm; numerous | |
| | | lenticles; | |
| 94 | Anther color | N79B | Greyed-purple |
| | | | N186 |
| 95 | Flower petal color | Yellow-green | Yellow-green |
| | | 145C | 145C |
| 96 | Style/Stigma description | Inconspicuous | Inconspicuous |

Botanical classification: Cornus kousa ‘Melissa's Mountain Snowfall’.

Unique Features: This tree features prolific flowering and exhibits fused bracts. About 80% of all bracts on the cultivar exhibit some degree of fusion (one side, two sides or three to four sides being fused), as shown in Table 5.

TABLE 5
Types of fused bracts observed on ‘Melissa's Mountain Bouquet’

| | Not | Two sides | 3 sides | Fully |
|---|---|---|---|---|
| Year | fused | fused | fused | Fused |
| 2016 | 29 | 23 | 17 | 32 |
| (n = 101) | (29%) | (23%) | (17%) | (32%) |
| 2017 | 39 | 28 | 33 | 45 |
| (n = 145) | (27%) | (19%) | (23%) | (31%) |
| 2019 | 7 | 12 | 14 | 90 |
| (n = 123) | (6%) | (10%) | (11%) | (73%) |
| Mean | 25 | 21 | 21 | 55.7 |
| | (20.7%) | (17.3%) | (17.0%) | (45.3%) |

Disease susceptibility: None noted. Powdery mildew caused by Erisphe pulchra was not observed. There was some minor occurrence of spot anthracnose caused by Elsinoe cornii observed in 2017-2019. Dogwood anthracnose caused by Discula destructiva has never been observed on ‘Melissa's Mountain Snowfall’.

Insect Damage: Minor insect damage on leaves.

### REFERENCES

Wadl, P. A., M. T. Windham, R. E. Evans, and R. N. Trigiano. 2014. Three new cultivars of Cornus kousa: Empire, Pam's Mountain Bouquet, and Red Steeple. HortScience 49(9):1230-1233.

We claim:

1. A new and distinct cultivar of Dogwood, Cornus kousa, named ‘MELISSA'S MOUNTAIN SNOWFALL’, as illustrated and described.
